(19) United States
(12) Patent Application Publication
(10) Pub. No.: US 2015/0150933 A1
(43) Pub. Date: Jun. 4, 2015

(54) METHOD AND COMPOSITIONS FOR IMPROVING A RESPONSE TO A METABOLIC STRESS

(71) Applicant: HIT SOUTHERN PTY LTD, Denistone, New South Wales (AU)

(72) Inventors: Jovan Hranisavljevic, Beograd (RS); Sergej Ostojic, Beograd (RS)

(73) Assignee: HIT SOUTHERN PTY LTD, Denistone, New South Wales (AU)

(21) Appl. No.: 14/404,850

(22) PCT Filed: May 3, 2013
(86) PCT No.: PCT/AU2013/000461

§ 371 Date: Dec. 1, 2014

(30) Foreign Application Priority Data
May 30, 2012 (RS) P-2012/0224

(51) Int. Cl. A61K 36/8998

(52) U.S. Cl. CPC A61K 36/8998

(57) ABSTRACT

The present disclosure relates to methods and compositions for improving a response to a metabolic stress. Certain embodiments provide a method of improving a response to a metabolic stress in a subject. The method comprises administering to a mucosal membrane of the subject an effective amount of an autolysate from rootlets of a barley plant of the species Hordeum, and/or an extract or a purified component of the autolysate.

### PRIORITY CLAIM

This application claims priority to Serbian patent application 20120224 (II-2012/0224) filed on 30 May 2012, the content of which is hereby incorporated by reference in its entirety.

### FIELD

The present disclosure relates to methods and compositions for improving a response to a metabolic stress.

### BACKGROUND

Metabolic stress is a condition that arises in response to an organism being subjected to increased metabolic demands, such as increased energetic demand, increased metabolic demand due to sickness, accelerated growth and tissue regeneration.

Metabolic stress also occurs as a response to strenuous physical activity in humans and animals. A variety of different types of strenuous physical activity can result in metabolic stress. For example, whilst training has a number of different positive benefits, such as increased physical capacity, there are a number of negative effects on the body, particularly when training occurs over extended periods of time. Over-training is a condition that arises after large increases in training load and/or volume, as well as during extremely prolonged activities, such as marathon racing or triathlon competition. In these cases, the physical activity may lead to a depression of the immune system. For example, endurance athletes are known to be particularly prone to cold and other upper respiratory infections after exhaustive exercise.

In addition, during and after exercise, the body releases the hormone cortisol into the bloodstream in response to stress. Cortisol is a catabolic hormone, which means that it degrades tissue, cellular structures and metabolically active molecules. While the release of cortisol is part of the body's normal response to stress, high levels of cortisol in the blood stream for long periods can lead to skeletal muscle proteins wastage and muscle mass reduction.

It has traditionally been accepted that dietary supplementation of most biomolecules and their precursors is not required, because the body is able to produce the majority of biomolecules from the various components present in food. For example, nucleotides are considered non-essential biomolecules as the components required for their synthesis are found in food.

However, it has become apparent that metabolic stress may provoke an increased demand for a variety of biomolecules, since the human body is not able to satisfy the increased needs from endogenous synthesis. For example, studies have indicated that that under metabolic stress an organism is not able to satisfy its physiological needs for nucleotides without an additional intake, and a recent study has indicated that oral dietary nucleotide supplementation may provide an improvement in an immunological marker (IgA) and a lowering in cortisol levels in subjects subjected to short term, high intensity exercise.

There is a large variety of oral nutritional supplements available, including many oral supplements in the form of tablets or capsules which are marketed for improving the response to physical stress. For example, nucleotide preparations are available for athletes which are a mixture of inactivated yeast cells and yeast extract. However, the efficacy of such supplements for improving the response to metabolic stress is unknown.

Accordingly, there is a need for methods and compositions for improving the response to metabolic stress.

### SUMMARY

The present disclosure relates to methods and compositions for improving a response to a metabolic stress.

Certain embodiments of the present disclosure provide a method of improving a response to a metabolic stress in a subject, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of improving physical performance in a subject, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of improving recovery from a metabolic stress in a subject suffering from or susceptible to a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of improving recovery from physical exercise in a subject, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of increasing immune function in a subject suffering from or susceptible to a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of increasing physical performance in a subject suffering from or susceptible to a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide a method for metabolic modulation in an athlete, the method comprising administering to a mucosal membrane of the athlete an effective amount of an autolysate from rootlets of a barley plant of the species Hordeum, and/or an extract or a purified component of the autolysate, wherein the administration improves endurance, decreases fatigue indices during and/or after physical exercise, provides faster recovery after maximal or sub-maximal activity, and/or provides stabilization of the immune system.

Certain embodiments of the present disclosure provide use of a barley rootlet autolysate of the species Hordeum and/or an extract or a purified component thereof in the preparation of a composition for mucosal administration to a subject to improve a response to a metabolic stress in the subject.

Certain embodiments of the present disclosure provide a composition for mucosal absorption, the composition comprising a barley rootlet autolysate of the species Hordeum and/or an extract or a purified component thereof.

Certain embodiments of the present disclosure provide a composition comprising a barley rootlet autolysate of the species Hordeum and/or an extract or a purified component thereof and one or more of a sweetener, a preservative and a stabiliser.

Certain embodiments of the present disclosure provide a method of improving a response to a metabolic stress in a subject, the method comprising administering to a mucosal membrane of the subject an effective amount of one or more nucleotides, nucleosides and products of the breakdown of nucleic acids, wherein one or more of the one or more nucleotides, nucleosides and products of the breakdown of nucleic acids are derived from a barley rootlet autolysate of the species Hordeum.

Certain embodiments of the present disclosure provide use of a composition comprising one or more nucleotides, nucleosides and products of the breakdown of nucleic acids in the preparation of a composition for mucosal administration to a subject to improve a response to a metabolic stress in the subject, wherein the composition is derived from a barley rootlet autolysate of the species Hordeum.

Certain embodiments of the present disclosure provide a composition comprising one or more nucleotides, nucleosides and products of the breakdown of nucleic acids, wherein the one or more of the one or more nucleotides, nucleosides and products of the breakdown of nucleic acids are derived from a barley rootlet autolysate of the species Hordeum.

Certain embodiments of the present disclosure provide a composition comprising the following components:
(i) one or more nucleotides, nucleosides, products of the breakdown of nucleic acids, and one or more plant hormones including kinetin, zeatin, an auxin and gibberellic acid, one or more of the aforementioned being derived from a barley rootlet autolysate of the species Hordeum; and
(ii) one or more of a sweetener, a preservative, a stabiliser and a gelling agent.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows a schematic of one embodiment of the extractive process of the present disclosure in which extracted biologically active compounds may be increased by recycling the product of the enzymatic extraction into a subsequent extraction process.

### DETAILED DESCRIPTION

The present disclosure relates to methods and compositions to improve a response to a metabolic stress.

Certain disclosed embodiments provide methods, compositions, and use of compositions that have one or more advantages. For example, some of the advantages of certain embodiments disclosed herein include one or more of the following: to improve a response to a metabolic stress in a subject; to improve recovery from a metabolic stress in a subject; to improve physical performance in a subject; to increase endurance in a subject; to decrease fatigue in a subject; to increase time to exhaustion in a subject; to improve recovery from training in a subject; to increase ventilation parameters in a subject; to decrease blood lactate levels in a subject; to improve immune function in a subject; to increase one or more immunological markers in a subject; to increase glucose transport in a subject; to decrease cortisol levels in a subject following exercise induced metabolic stress; to provide improved nutraceutical compositions; to provide athletes with compositions to improve one or more of physical performance, reduce fatigue and/or recovery from exercise; and to provide dietary supplements with improved properties. Other advantages of certain embodiments of the present disclosure are disclosed herein.

The present disclosure is based, at least in part, upon the recognition mucosal delivery of a barley rootlet autolysate (and/or an extract or purified component thereof) provides improved properties, and in particular improves a variety of physical performance parameters, recovery and immune function in athletes. Without being bound by theory, it has been recognised that components in an autolytic extract provide a number of beneficial properties when delivered via the mucosal route as compared to the digestive route, as components in an autolysate are susceptible to enzymatic destruction while passing through the digestive system.

For example, administration of an autolysate from rootlets of a barley plant of the species Hordeum (and/or an extract or a purified component of the autolysate) to a mucosal membrane of an athlete may result in metabolic modulation that provides one or more beneficial effects including improving endurance, decreasing fatigue indices during and/or after physical exercise, providing faster recovery after maximal or sub-maximal activity, and/or providing stabilization of the immune system.

Certain embodiments of the present disclosure provide a method of improving a response to a metabolic stress in a subject using an autolysate (and/or an extract or a purified component of the autolysate) delivered by a mucosal route, as described herein.

Certain embodiments of the present disclosure provide a method of improving a response to a metabolic stress in a subject, the method comprising administering to a mucosal membrane of the subject an effective amount of an autolysate from rootlets of a barley plant of the species Hordeum, and/or an extract or a purified component of the autolysate.

In certain embodiments, the metabolic stress comprises exercise induced metabolic stress. In certain embodiments, the metabolic stress comprises over-training induced metabolic stress.

In certain embodiments, the metabolic stress comprises an increased state of energetic and/or metabolic demand, an increased energetic and/or metabolic demand due to physical activity or exercise, an increased energetic and/or metabolic demand due to sickness, an increased energetic and/or metabolic demand due to accelerated growth, and an increased energetic and/or metabolic demand due to tissue regeneration. Other metabolic stresses are contemplated.

Certain embodiments of the present disclosure provide a method of improving a response to exercise induced metabolic stress in a subject using an autolysate (and/or an extract or a purified component of the autolysate) delivered by a mucosal route, as described herein.

Certain embodiments of the present disclosure provide a method of improving a response to exercise induced metabolic stress in a subject, the method comprising administering to a mucosal membrane of the subject an effective amount of an autolysate from rootlets of a barley plant of the species Hordeum, and/or an extract or a purified component of the autolysate.

In certain embodiments, the response to the metabolic stress comprises one or more of metabolic modulation, physical performance, endurance, fatigue, time to exhaustion, recovery from training, ventilation parameters, blood lactate levels, immune function, one or more immunological markers, glucose transport, and cortisol levels in a subject following exercise induced metabolic stress.

In certain embodiments, the response to a metabolic stress comprises physical performance.

Certain embodiments of the present disclosure provide a method of improving physical performance in a subject using an autolysate (and/or an extract or a purified component of the autolysate) delivered by a mucosal route, as described herein.

Certain embodiments of the present disclosure provide a method of improving physical performance in a subject, the method comprising administering to a mucosal membrane of the subject an effective amount of an autolysate from rootlets of a barley plant of the species Hordeum, and/or an extract or a purified component of the autolysate.

In certain embodiments, the response to a metabolic stress comprises recovery to a metabolic stress.

Certain embodiments of the present disclosure provide a method of improving recovery from a metabolic stress in a subject using an autolysate (and/or an extract or a purified component of the autolysate) delivered by a mucosal route, as described herein.

Certain embodiments of the present disclosure provide a method of improving recovery from a metabolic stress in a subject, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of improving recovery from physical exercise in a subject using an autolysate (and/or an extract or a purified component of the autolysate) delivered by a mucosal route, as described herein.

Certain embodiments of the present disclosure provide a method of improving recovery from physical exercise in a subject, the method comprising administering to a mucosal membrane of the subject an effective amount of an autolysate from rootlets of a barley plant of the species Hordeum, and/or an extract or a purified component of the autolysate.

In certain embodiments, the metabolic stress comprises over-training induced metabolic stress.

Certain embodiments of the present disclosure provide a method of improving recovery from exercise induced metabolic stress and/or over-training induced metabolic stress in a subject using an autolysate (and/or an extract or a purified component of the autolysate) delivered by a mucosal route, as described herein.

Certain embodiments of the present disclosure provide a method of improving recovery from exercise induced metabolic stress and/or over-training induced metabolic stress in a subject, the method comprising administering to a mucosal membrane of the subject an effective amount of an autolysate from rootlets of a barley plant of the species Hordeum, and/or an extract or a purified component of the autolysate.

In certain embodiments, the subject of the present disclosure is a human subject. In certain embodiments, the subject is an animal subject. In certain embodiments, the subject is a mammalian subject, a livestock animal (such as a horse, a cow, a sheep, a goat, a pig), a domestic animal (such as a dog or a cat) and other types of animals such as monkeys, rabbits, mice and laboratory animals. Other types of animals are contemplated. Veterinary applications of the present disclosure are contemplated.

In certain embodiments, the subject is a human or an animal. In certain embodiments, the subject is an athlete.

In certain embodiments, the subject is susceptible to, or suffering from, a metabolic stress. Examples of metabolic stresses are as described herein.

Certain embodiments of the present disclosure provide a method of improving a response to a metabolic stress in a subject susceptible to or suffering from a metabolic stress using an autolysate (and/or an extract or a purified component of the autolysate) delivered by a mucosal route, as described herein.

Certain embodiments of the present disclosure provide a method of improving a response to a metabolic stress in a subject susceptible to or suffering from a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide a method of increasing physical performance in a subject susceptible to or suffering from a metabolic stress using an autolysate (and/or an extract or a purified component of the autolysate) delivered by a mucosal route, as described herein.

Certain embodiments of the present disclosure provide a method of increasing physical performance in a subject susceptible to or suffering from a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide method of improving recovery from a metabolic stress in a subject using an autolysate (and/or an extract or a purified component of the autolysate) delivered by a mucosal route, as described herein.

In certain embodiments, the subject is suffering from or susceptible to a metabolic stress.

Certain embodiments of the present disclosure provide method of improving recovery from a metabolic stress in a subject suffering from or susceptible to a metabolic stress, the method comprising administering to a mucosal membrane of the subject an effective amount of an autolysate from rootlets of a barley plant of the species Hordeum, and/or an extract or a purified component of the autolysate.

Certain embodiments of the present disclosure provide a method for metabolic modulation in a subject using an autolysate (and/or an extract or a purified component of the autolysate) delivered by a mucosal route, as described herein.

Certain embodiments of the present disclosure provide a method for metabolic modulation in subject, the method comprising administering to a mucosal membrane of the subject an effective amount of an autolysate from rootlets of a barley plant of the species Hordeum, and/or an extract or a purified component of the autolysate, wherein the administration improves endurance, decreases fatigue indices during and/or after physical exercise, provides faster recovery after maximal or sub-maximal activity, and/or provides stabilization of the immune system.

Certain embodiments of the present disclosure provide a method for metabolic modulation in an athlete using an autolysate (and/or an extract or a purified component of the autolysate) delivered by a mucosal route, as described herein.

Certain embodiments of the present disclosure provide a method for metabolic modulation in an athlete, the method comprising administering to a mucosal membrane of the athlete an effective amount of an autolysate from rootlets of a barley plant of the species Hordeum, and/or an extract or a purified component of the autolysate, wherein the administration improves endurance, decreases fatigue indices during and/or after physical exercise, provides faster recovery after maximal or sub-maximal activity, and/or provides stabilization of the immune system.

Certain embodiments of the present disclosure provide a method of increasing immune function in a subject using an autolysate (and/or an extract or a purified component of the autolysate) delivered by a mucosal route, as described herein.

In certain embodiments, the subject is suffering from or susceptible to a metabolic stress.

Certain embodiments of the present disclosure provide a method of increasing immune function in a subject suffering from or susceptible to a metabolic stress, the method comprising administering to a mucosal membrane of the subject an effective amount of an autolysate from rootlets of a barley plant of the species Hordeum, and/or an extract or a purified component of the autolysate.

In certain embodiments, the barley plant of the present disclosure comprises a domesticated barley plant. In certain embodiments, the barley plant comprises a wild barley plant.

In certain embodiments, the barley plant comprises a plant of the Hordeum species and/or a sub-species, variety, form or cultivar thereof. In certain embodiments, the barley plant comprises a Hordeum vulgare L. plant, and/or a sub-species, variety, form or cultivar thereof.

Autolysis is a process of self digestion and/or breakdown of a product, including, for example, hydrolysis of a variety of macromolecular species such as nucleic acids and/or proteins, producing amongst others low molecular structural elements such as oligonucleotides, nucleotides, nucleosides, peptides, amino acids. Autolysis may also result in the release plant hormones from their complexing agents. Typically, the autolysate is a complex mixture of biomolecules.

The term “barley rootlets” refers to one or more side products from the barley malting process containing external parts of malted grains removed after kilning the malt with deculmer (a rootlet removing device). Typically, rootlets will also include sprouts and husks.

In certain embodiments, barley rootlets represent a mixture of rootlets, and one or more of sprouts and husks, which have been separated from the malted barley kernel in the brewing process. Typically, a barley rootlets autolysate contains a complex mixture of biomolecules coming from tissues of sprouted seed, such as rootlets and sprouts, as well as from husks. An autolysate may contain nucleotidic components, such as nucleotides, nucleosides and cytokinins (plant growth hormones); amino acids, essential and non-essential, as well as numerous non-identified substances from plant stem cells.

Methods for producing an autolysate are known in the art and are described herein.

In certain embodiments, the present disclosure provides use of an extract and/or a purified component of an autolysate of rootles of a barley plant. The term extract includes, for example, any fraction, preparation, purified or semi purified component, or concentrate derived from an autolysate. For example, the extract may be a complex mixture of plant constituents or a fraction resulting from the concentration, purification or partitioning of one or more active ingredients present in the complex mixture. The tern “purified component” includes, for example, any fraction, preparation, or purified or semi purified component of an autolysate.

In certain embodiments, the autolysate comprises an autolysate derived from rootlets produced from malting.

In certain embodiments, the autolysate comprises an autolysate from sprouts and/or husks.

In certain embodiments the autolysate (and/or an extract or purified component thereof) may contain components that activate the enzyme 5′-adenosine-monophosphate kinase (AMPK) and/or increase the number of glucose transporting proteins (for example GLUT—4), thereby pointing to a non-insulin metabolic pathway of glucose to cells. In certain embodiments, facilitated transport of glucose to muscle cells may lead to the production of cellular energetic reserves in the form of glycogen, which may be convenient for athletes to provide enough energy during long sporting efforts.

In certain embodiments, the autolysate and/or the extract or purified component comprises one or more nucleotides, nucleosides and products of the breakdown of barley nucleic acids. Methods for enriching and/or purifying one or more of nucleotides, nucleosides and products of the breakdown of nucleic acids from mixtures are known in the art.

In certain embodiments, the one or more nucleotides, nucleosides and products of the breakdown of barley nucleic acids comprises a molecular weight of less than 1000 Da.

In certain embodiments, the autolysate and/or the extract or purified component comprises one or more plant hormones. Examples include cytokinins (kinetin, zeatin and 6-benzylaminopurine), auxins and gibberellic acid. Methods for enriching and/or purifying plant hormones are known in the art.

In certain embodiments, the autolysate and/or the extract or purified component thereof comprises one or more amino acids and/or a modulator of carbohydrate metabolism. Methods for enriching and/or purifying amino acids and/or modulators of carbohydrate metabolism are known in the art.

In certain embodiments, the mucosal membrane comprises an oral mucosal membrane. In certain embodiments, the mucosal membrane comprises one or more of a buccal mucosal membrane, a sublingual mucosal membrane, a palatal mucosal membrane, a gingival mucosal membrane and a labial mucosal membrane.

In certain embodiments, the administering comprises oral mucosal administration. In certain embodiments, the mucosal membranes in the mouth cavity may be used, enabling direct transport to bloodstream and facilitating high efficacy of application.

In certain embodiments, the autolysate (and/or an extract or purified component thereof) are formulated so as to be suitable for transport to an organism by oral mucosal membranes, such as solutions suitable for spraying into the mouth cavity (spray), jellylike forms suitable for suckling (jelly) which dissolve or disperse in saliva, candies, lingvaletes and other mouth releasing forms known in the art, and chewing gums.

In certain embodiments, the autolysate and/or an extract or a purified component thereof is administered in a form comprising one or more of a solution, a spray, a gel, a jelly, a candy, an oral disintegrating tablet, and a gum. Methods for formulating compositions for oral mucosal delivery are known in the art.

In certain embodiments, the autolysate and/or an extract or purified component thereof is administered once a day, twice a day or three times a day. In certain embodiments, the autolysate and/or an extract or purified component thereof is administered at least once daily, at least twice daily, or at least three times daily.

In certain embodiments, the method comprises at least once daily administration, at least twice daily administration, or at least three times daily administration.

In certain embodiments, the method comprises a combined daily administration of autolysate solution (for example as described in Example 4) in an amount of least 400 mg, at least 500 mg, at least 600 mg, at least 700 mg, at least 800 mg, at least 900 mg or at least 1 g. Other amounts are contemplated.

In certain embodiments, the method comprises a combined daily administration of autolysate solution in an amount of 400 mg to 1600 mg, 400 to 1500 mg, 400 to 1300 mg, 400 to 1300 mg, 400 to 1200 mg, 400 to 1100 mg, 400 to 1000 mg, 400 to 900 mg, 400 to 800 mg, 400 to 700 mg, 400 to 600 mg, 400 to 500 mg, 500 mg to 1600 mg, 500 to 1500 mg, 500 to 1300 mg, 500 to 1300 mg, 500 to 1200 mg, 500 to 1100 mg, 500 to 1000 mg, 500 to 900 mg, 500 to 800 mg, 500 to 700 mg, 500 to 600 mg, 600 mg to 1600 mg, 600 to 1500 mg, 600 to 1600 mg, 600 to 1300 mg, 600 to 1200 mg, 600 to 1100 mg, 600 to 1000 mg, 600 to 900 mg, 600 to 800 mg, 600 to 700 mg, 700 mg to 1600 mg, 700 to 1500 mg, 700 to 1400 mg, 700 to 1300 mg, 700 to 1200 mg, 700 to 1100 mg, 700 to 1000 mg, 700 to 900 mg, 700 to 800 mg, 800 mg to 1600 mg, 800 to 1500 mg, 800 to 1400 mg, 800 to 1300 mg, 800 to 1200 mg, 800 to 1100 mg, 800 to 1000 mg, 800 to 900 mg, 900 mg to 1600 mg, 900 to 1500 mg, 900 to 1400 mg, 900 to 1300 mg, 900 to 1200 mg, 900 to 1100 mg, 900 to 1000 mg, 1000 mg to 1600 mg, 1000 to 1500 mg, 1000 to 1400 mg, 1000 to 1300 mg, 1000 to 1200 mg, 1000 to 1100 mg, 1100 mg to 1600 mg, 1100 to 1500 mg, 1100 to 1400 mg, 1100 to 1300 mg, 1100 to 1200 mg, 1200 mg to 1600 mg, 1200 to 1500 mg, 1200 to 1400 mg, 1200 to 1300 mg, 1300 mg to 1600 mg, 1300 to 1500 mg, 1300 to 1400 mg, or 1400 to 1500 mg. Other amounts are contemplated.

In certain embodiments, the method comprises a combined daily administration of dry autolysate powder (as described for example in Example 3) in an amount of least 10 mg, at least 15 mg, at least 20 mg, at least 25 mg, at least 30 mg, at least 35 mg or at least 40 mg. Other amounts are contemplated.

In certain embodiments, the method comprises a combined daily administration of dry autolysate powder (as described for example in Example 3) in an amount of 10 to 40 mg, 10 to 35 mg, 10 to 30 mg, 10 to 25 mg, 10 to 20 mg, 10 to 15 mg, 15 to 40 mg, 15 to 35 mg, 15 to 30 mg, 15 to 25 mg, 15 to 20 mg, 20 to 40 mg, 20 to 35 mg, 20 to 30 mg, 20 to 25 mg, 25 to 40 mg, 25 to 35 mg, 25 to 30 mg, 30 to 40 mg, 30 to 35 mg, or 35 to 40 mg. Other amounts are contemplated.

In certain embodiments, the method comprises a combined daily administration of dry autolysate powder (as described for example in Example 3) in an amount of least 10 μg/kg body weight, at least 15 μg/kg, at least 20 μg/kg, at least 25 μg/kg, at least 30 μg/kg, at least 35 μg/kg, at least 40 μg/kg, at least 45 μg/kg, at least 50 μg/kg, at least 55 μ/kg, or at least 60 μ/kg body weight. Other amounts are contemplated.

In certain embodiments, the method comprises a combined daily administration of dry autolysate powder (see Example 3) in an amount of 10 to 60 μg/kg body weight, 10 to 55 μg/kg, 10 to 50 μg/kg, 10 to 45 μg/kg 10 to 40 μg/kg, 10 to 35 μg/kg, 10 to 30 μg/kg, 10 to 25 μg/kg, 10 to 20 μg/kg, 10 to 15 μg/kg, 15 to 60 μg/kg body weight, 15 to 55 μg/kg, 15 to 50 μg/kg, 15 to 45 μg/kg, 15 to 40 μg/kg, 15 to 35 μg/kg, 15 to 30 μg/kg, 15 to 25 μg/kg, 15 to 20 μg/kg, 20 to 60 μg/kg body weight, 20 to 55 μg/kg, 20 to 50 μg/kg, 20 to 45 μg/kg, 20 to 40 μg/kg, 20 to 35 μg/kg, 20 to 30 μg/kg, 20 to 25 μg/kg, 25 to 60 μg/kg body weight, 25 to 55 μg/kg, 25 to 50 μg/kg, 25 to 45 μg/kg, 25 to 40 μg/kg, 25 to 35 μg/kg, 25 to 30 μg/kg, 30 to 60 μg/kg, 30 to 55 μg/kg, 30 to 50 μg/kg, 30 to 45 μg/kg, 30 to 40 μg/kg, 30 to 35 μg/kg, 35 to 60 μg/kg body weight, 35 to 55 μg/kg, 35 to 50 μg/kg, 35 to 45 μg/kg, 35 to 40 μg/kg, 40 to 60 μg/kg body weight, 40 to 55 μg/kg, 40 to 50 μg/kg, 45 to 60 μg/kg body weight, 45 to 55 μg/kg, 45 to 50 μg/kg, 50 to 60 μg/kg body weight, 50 to 55 μg/kg, or 55 to 60 μg/kg body weight. Other amounts are contemplated.

For example, one administration daily with 10 sprays of a composition as prepared in Example 4 into the mouth cavity may be used. This equates to 1.25-1.30 g. of the preparation containing approximately 830-850 mg autolysate solution or approx. 21 mg dry matter or approximately 26 μg/kg BWM of extracted dry matter.

In certain embodiments, the autolysate and/or an extract or purified component thereof is administered prior to the metabolic stress. In certain embodiments, the autolysate and/or an extract or purified component thereof is administered after a metabolic stress. In certain embodiments, the autolysate and/or an extract or purified component thereof is administered concurrently and/or after a metabolic stress.

In certain embodiments, the autolysate and/or an extract or purified component thereof is administered for at least 7 days. In certain embodiments, the autolysate and/or an extract or purified component thereof is administered for at least 14 days. In certain embodiments, the autolysate and/or an extract or purified component thereof is administered for at least 1 month, at least 2 months, or at least 3 months. In certain embodiments, autolysate and/or an extract or purified component thereof is administered on a perpetual or continuing basis.

In certain embodiments, the mucosal membrane comprises a nasal mucosal membrane. In certain embodiments, the administering comprises nasal administration.

In certain embodiments, the autolysate and/or an extract or a purified component thereof is administered nasally in a form comprising one or more of a solution, a spray, or a gel. Methods for formulating compositions for nasal mucosal delivery are known in the art.

In certain embodiments, the methods of the present disclosure are used to improve physical performance in the subject, to increase endurance in the subject, to increase general aerobic endurance in the subject, to decrease fatigue in the subject, to decrease fatigue indices during and/or after physical exercise, to increase time to exhaustion in the subject, to improve recovery from training in the subject, for faster recovery after maximal or sub-maximal activity, to increase ventilation parameters in the subject, to decrease blood lactate levels in the subject, to improve immune function in the subject, to stabilise the immune system in the subject, to increase one or more immunological markers in the subject, to increase glucose transport in the subject, to decrease cortisol levels in a subject following exercise induced metabolic stress, for metabolic modulation, for metabolic modulation in athletes during sport activities in order to increase general (aerobic) endurance and decrease fatigue indices during and after physical exercises, provide faster recovery after maximal and sub-maximal activities, and/or stabilization of the immune system.

In certain embodiments, the autolised barley rootlets extract is produced from a side product of barley malting process, and comprises a mixture of rootlets, sprouts and husk.

In certain embodiments, the autolysate (and/or an extract or purified component thereof) comprises active modulators of carbohydrate metabolism present in eukaryotic cells.

In certain embodiments, the autolysate (and/or an extract or purified component thereof) comprises active substances characterized with a low-molecular weight and a nucleotide structure (less than 1000 Da).

In certain embodiments, the autolysate (and/or an extract or purified component thereof) comprises nucleotide substances that exhibit activity advantageous to athletes.

In certain embodiments, the autolysate (and/or an extract or purified component thereof) comprises an effect on the ultra-fast recovery to athletes after maximal efforts.

In certain embodiments, the autolysate (and/or an extract or purified component thereof) comprises an effect relating to indices of recovery after physical efforts, including heart frequency, dynamics of blood lactate concentration and ventilation parameters.

In certain embodiments, the autolysate (and/or an extract or purified component thereof) comprises an effect relating to stabilization of immunological functions of athletes liable to changes at maximal physical effort.

In certain embodiments, the autolysate (and/or an extract or purified component thereof) comprises an effect characterized in the increase of serum immunomodulatory components, such as immunoglobulin A, natural killer cells as well as cytotoxic activity.

In certain embodiments, the autolysate (and/or an extract or purified component thereof) comprises an effect characterized in stabilizing activity on the salivary immunoglobulins A and M levels during physical effort.

In certain embodiments, the autolysate (and/or an extract or purified component thereof) is convenient for preparation of ready-made forms for application in the mouth cavity to the oral mucosal membranes.

In certain embodiments, the autolysate (and/or an extract or purified component thereof) is suitable for preparation in a liquid form suitable for dispersion in the mouth cavity by spraying.

In certain embodiments, the autolysate (and/or an extract or purified component thereof) comprises a gelatinous structure suitable for dispersion and solution in the mouth by saliva.

Certain embodiments of the present disclosure provide use of a barley rootlet autolysate of the species Hordeum and/or an extract or a purified component thereof in the preparation of a composition.

Certain embodiments of the present disclosure provide use of a barley rootlet autolysate of the species Hordeum and/or an extract or a purified component thereof in the preparation of a composition for mucosal administration to a subject to improve a response to a metabolic stress in the subject.

Certain embodiments of the present disclosure provide preparations for metabolic modulation in athletes to increase general (aerobic) endurance performance and/or decrease fatigue during and after physical exercises, and/or enhanced recovery from maximal and/or sub maximal activities.

Certain embodiments of the present disclosure provide preparations from malted barley rootlets as side products in beer production. In certain embodiments, these preparations include biologically active substances that beneficially influence metabolism.

Certain embodiments of the present disclosure provide a composition for mucosal absorption.

Certain embodiments of the present disclosure provide a composition for mucosal absorption, the composition comprising a barley rootlet autolysate of the species Hordeum and/or an extract or a purified component thereof.

In certain embodiments, the mucosal absorption comprises oral mucosal absorption. In certain embodiments, the composition comprises a composition for oral mucosal absorption.

In certain embodiments, the mucosal absorption comprises one or more of buccal mucosal absorption, sublingual mucosal absorption, palatal mucosal absorption, gingival mucosal absorption and a labial mucosal absorption. In certain embodiments, the composition comprises a composition for one or more of buccal mucosal absorption, sublingual mucosal absorption, palatal mucosal absorption, gingival mucosal absorption and a labial mucosal absorption.

In certain embodiments, the composition is in a form comprising one or more of a solution, a spray, a gel, a jelly, a candy, an oral disintegrating tablet and a gum. Methods for formulating such compositions are known in the art. Methods for the preparation of compositions are known, and are as described, for example, in Remington's Pharmaceutical Sciences, 18th ed., 1990, Mack Publishing Co., Easton, Pa. and U.S. Pharmacopeia: National Formulary, 1984, Mack Publishing Company, Easton, Pa.

Compositions may include an oral dissolution agent to enhance the delivery or release of the autolysate and/or an extract or a purified component thereof. Suitable oral dissolution agents include, for example, commonly used and accepted pharmaceutical ingredients, such as sugars or sweeteners, saccharides, carbohydrates, polymers, excipients, and the like, capable of breaking down in and/or dissolving in fluids of the oral cavity. Examples of suitable oral dissolution agents include, without limitation, acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, poly oxyethylene alkyl ethers, polyvinyl alcohol, propylene glycol alginate, sodium alginate, sodium ascorbate, sodium starch glyolate, sodium saccharin, sorbitol, starch, pregelatinized starch, sucrose, tragacanth, trimethylglycine, xanthan gum, xylitol, zein, and combinations thereof.

The compositions may also include a pH buffer. Examples of suitable pH buffers include at least one of a phosphate buffer, a glycylglycine buffer, a carbonate buffer, a bicarbonate buffer, a tris buffer, a tartrate buffer, a borate buffer, an acetate buffer, and a maleate buffer. Combinations of buffers may be utilized to obtain the desired pH in the oral cavity.

The composition may further include additional ingredients to provide desirable characteristics, such as aesthetically pleasing qualities, improved taste, and the like, to otherwise render the dosage formulation more likely to be administered by the patient. Examples of such ingredients include, absorbants, colorants, flavorants, solvents and co-solvents, coating agents, direct compression excipients, disintegrants, glidants, lubricants, opaquants, polishing agents, suspending agents, sweetening agents, anti-adherents, binders, and capsule diluents. The ingredients may also include anti-fungal preservatives, anti-microbial preservatives, clarifying agents, emulsifying agents, antioxidants, levigating agents, plasticizers, surfactants, tonicity agents, viscosity increasing agents and combinations thereof. Examples of useful additives include propylene glycol, polyethylene glycol (PEG), orange, cherry, and strawberry flavors, stevia powder, and other commonly utilized ingredients.

The components of the composition may be formulated in any suitable dosage form. For example, suitable formulations include solid formulations such as a lozenge, a lollipop, a troche, a dragee, a chewable gum, a solid candy, a granular solid, a chewable tablet or pill, an orally dispersable tablet or pill, an orally dissolvable tablet, an orally dissolvable pill and an orally dissolvable capsule. In one embodiment, the composition is formulated as one of a lollipop and a lozenge. Alternatively, the formulation may be a liquid formulation, including, a solution, a suspension, and an emulsion. Such formulations may be prepared utilizing formulating procedures known in this art. For example, there are several ways to create a solid, orally dissolvable formulation, including wet granulation, co-melt, spray-drying, freeze-drying, and the like. Particularly, solid formulations such as lozenges, solid candies, lollipops, or lozenges on a stick, and the like may be prepared utilizing such techniques, including wet granulation, co-melt, spray-drying, freeze-drying, and the like. Solid formulation may also be made by a partial wet-granulation process.

In certain embodiments, the mucosal absorption comprises nasal mucosal absorption. In certain embodiments, the composition comprises a composition for nasal mucosal absorption.

In certain embodiments, the composition is in a form comprising a solution or a spray. Methods for formulating compositions for nasal mucosal administration are known in the art.

The autolysate and/or an extract and/or purified component thereof are as described herein.

In certain embodiments, the autolysate and/or the extract or purified component thereof comprises one or more nucleotides, nucleosides and products of the breakdown of barley nucleic acids, as described herein. In certain embodiments, the one or more nucleotides, nucleosides and products of the breakdown of barley nucleic acids comprises a molecular weight of less than 1000 Da, as described herein.

In certain embodiments, the autolysate and/or the extract or purified component comprises one or more plant hormones, as described herein.

In certain embodiments, the autolysate and/or the extract or purified component thereof comprises one or more amino acids and/or a modulator of carbohydrate metabolism, as described herein.

In certain embodiments, the autolysate and/or the extract or purified component thereof comprises one or more amino acids and/or a modulator of carbohydrate metabolism, as described herein.

In certain embodiments, the autolysate comprises an autolysate derived from rootlets produced from malting, as described herein.

In certain embodiments, the autolysate comprises an autolysate from sprouts and/or husks, as described herein.

In certain embodiments, the composition comprises one or more of a sweetener, a preservative, a stabiliser and a gelling agent, as described herein.

Certain embodiments of the present disclosure provide a method of improving a response to a metabolic stress in a subject, the method comprising administering a composition as described herein to a mucosal membrane of the subject.

Examples of responses to metabolic stress are as described herein.

Certain embodiments of the present disclosure provide a composition as described herein for use for one or more of the following: to improve physical performance in the subject, to increase endurance in the subject, to increase general aerobic endurance in the subject, to decrease fatigue in the subject, to decrease fatigue indices during and/or after physical exercise, to increase time to exhaustion in the subject, to improve recovery from training in the subject, for faster recovery after maximal or sub-maximal activity, to increase ventilation parameters in the subject, to decrease blood lactate levels in the subject, to improve immune function in the subject, to stabilise the immune system in the subject, to increase one or more immunological markers in the subject, to increase glucose transport in the subject, to decrease cortisol levels in a subject following exercise induced metabolic stress, for metabolic modulation, for metabolic modulation in athletes during sport activities in order to increase general (aerobic) endurance and decrease fatigue indices during and after physical exercises, provide faster recovery after maximal and sub-maximal activities, and/or stabilization of the immune system.

Certain embodiments of the present disclosure provide a composition comprising a barley rootlet autolysate of the species Hordeum (and/or an extract or a purified component thereof) and one or more additional ingredients, agents or additives as described herein.

Certain embodiments of the present disclosure provide a composition comprising a barley rootlet autolysate of the species Hordeum and/or an extract or a purified component thereof and one or more of a sweetener, a preservative and a stabiliser.

Certain embodiments of the present disclosure provide a method of improving a response to a metabolic stress in a subject, the method comprising administering to a mucosal membrane of the subject an effective amount of one or more nucleotides, nucleosides and products of the breakdown of nucleic acids, wherein one or more of the one or more nucleotides, nucleosides and products of the breakdown of nucleic acids are derived from a barley rootlet autolysate of the species Hordeum.

Certain embodiments of the present disclosure provide use of a composition comprising one or more nucleotides, nucleosides and products of the breakdown of nucleic acids.

Certain embodiments of the present disclosure provide use of a composition comprising one or more nucleotides, nucleosides and products of the breakdown of nucleic acids in the preparation of a composition for mucosal administration to a subject to improve a response to a metabolic stress in the subject, wherein the composition is derived from a barley rootlet autolysate of the species Hordeum.

In certain embodiments, the one or more nucleotides, nucleosides and products of the breakdown of nucleic acids derived from a barley rootlet autolysate of the species Hordeum are present in an extract or a purified component of the autolysate. Methods for enriching for nucleotides, nucleosides and products of the breakdown of nucleic acids are known in the art.

Certain embodiments of the present disclosure provide a composition comprising one or more nucleotides, nucleosides and products of the breakdown of nucleic acids, wherein the one or more of the one or more nucleotides, nucleosides and products of the breakdown of nucleic acids are derived from a barley rootlet autolysate of the species Hordeum.

Certain embodiments of the present disclosure provide a composition comprising one or more plant hormones including, kinetin, zeatin, an auxin and gibberellic acid, wherein the one or more plant hormones are derived from a barley rootlet autolysate of the species Hordeum.

In certain embodiments, the one or more plant hormones derived from a barley rootlet autolysate of the species Hordeum are in present in an extract or a purified component of the autolysate. Methods for enriching for one or more plant hormones are known in the art.

Certain embodiments of the present disclosure provide compositions comprising enriched components from an autolysate. Certain embodiments of the present disclosure provide use of compositions comprising enriched components from an autolysate in the preparation of a formulation for mucosal administration to a subject to improve a response to a metabolic stress in the subject.

Certain embodiments of the present disclosure provide a composition comprising the following components:
(i) one or more nucleotides, nucleosides and products of the breakdown of nucleic acids, one or more of the aforementioned being derived from a barley rootlet autolysate of the species Hordeum; and
(ii) one or more of a sweetener, a preservative, a stabiliser and a gelling agent.

Certain embodiments of the present disclosure provide a composition comprising the following components:
(i) one or more nucleotides, nucleosides, products of the breakdown of nucleic acids, and one or more plant hormones including kinetin, zeatin, an auxin and gibberellic acid, one or more of the aforementioned being derived from a barley rootlet autolysate of the species Hordeum; and
(ii) one or more of a sweetener, a preservative, a stabiliser and a gelling agent.

The present disclosure is further described by the following examples. It is to be understood that the following description is for the purpose of describing particular embodiments only and is not intended to be limiting with respect to the above description.

### Example 1

The following example provides a procedure for the production of the malted barley rootlets autolized extract and the distribution of biological activities present in different parts of malted barley tissues.

Brewers barley grain was malted in accordance with the methodology known in the art, and are as described in, for example, the following: Marcey, A (1963). Brew. Guardian 92, 41, Kellet, O. S. (1965). MBAA Tech. Q. 2, 69; Bradee, L. H., and Westermann, D. H. (1975), Internal communication, p. 37. Jos. Schlitz Brewing Co., Milwaukee, Wis., MICROBIAL TECHNOLOGY—Fermentation technology, Volume II. Academic Press Inc, Editors: H. J. Peppier and D. Perlman (1978), Chapter 1.: Beer Brewing (D. H. Westermann and N. J. Huige), Malting Process, p. 16-19, the contents of which are hereby incorporated by reference in their entirety.

The samples produced were determined to be samples of standard industrial production and the quality and composition were considered to be typical of standard commercial samples.

The sampling was performed at the final stage of malting, after kilning and removal of external parts, such as rootlets, sprouts and husks, from the malted grains.

Biological activity was evaluated with 3 types of samples:
1. Dried integral malted barley (“MB integral”)
2. MB grains depleted of external parts (“MB grains”)
3. Removed external parts (“MB rootlets”)

Comparative biological activity tests were based on “Yeast fermentation rate evaluation” using a modified Warburg method (Mirsky, N. et al., J. Inorg. Biochem. 13, 11-21, 1980). Sampling was performed from different batches during a malting campaign at 4 month intervals. The results are given at Table 1.

TABLE 1

| Sample | Activity (%) | Mean value (%) | Samples No (n) |
|---|---|---|---|
| 1. MB integral | 18.9-26.2 | 22.2 | 4 |
| 2. MB grains | 16.1-17.3 | 16.7 | 3 |
| 3. MB rootlets | 30.7-66.7 | 42.7 | 8 |

The yeast fermentation comparative rates clearly point to a disproportion in the location of active molecules in malted barley compartments, with a great advantage present in MB rootlets.

These results also indicate that the advantageous biological activity located in the rootlets originate from the presence of plant stem cells in that tissue, contributing to the additional presence of active components such as nucleotides, nucleosides, nucleic acid breakdown products, plant hormones, such as cytokinins and other active biomolecules.

### Example 2

The following example shows the dependence of extract activity level on time of autolysis.

Samples of ground MB rootlets powder in a quantity of 150 mg were suspended in 5 ml water in a sealed vessel of 15 ml, and submitted to auto digestion in a thermostat at 36° C. at time intervals from 6 to 24 h. Digested samples were quantitatively transferred to analytical fermenters and evaluated for biological activity for yeast cell fermentation rate in comparison to a control.

The results are shown at Table 2.

TABLE 2

| Time (h) | Activity (%) |
|---|---|
| 6 | 48 |
| 9 | 52 |
| 14 | 64.4 |
| 24 | 60 |

The results demonstrate that a time period of 14 h is optimal for the release of active biomolecules from macromolecular complex structures and facilitate its biological availability.

### Example 3

The following example provides a procedure for the production of the autolised extract of the present disclosure.

MB rootlets (15 g.) obtained after MB kilning were suspended in 200 ml tap water and digested at 36° C. for 14 h with occasional stirring.

After digestion the suspension was centrifuged at a rotation speed of 800 r/min 110 ml of clear supernatant was obtained while an additional 90 ml of solution remained bound to the fibrous pulp, and proportional aliquot of the active solutes.

The pulp was resuspended in 100 ml of water and after frequent stiffing and centrifugation gave an additional 90 ml of extract. The fractions were combined to give a total 200 ml of product in the form of solution, which is convenient for the preparation of liquid preparations.

This process allows enzymatic action to take place during the incubation process, which results in a crude liquor which contains a complex mixture of nucleotides, nucleosides, nucleic acid breakdown products, cytokinins, other plant hormones, amino acids and other biomolecules

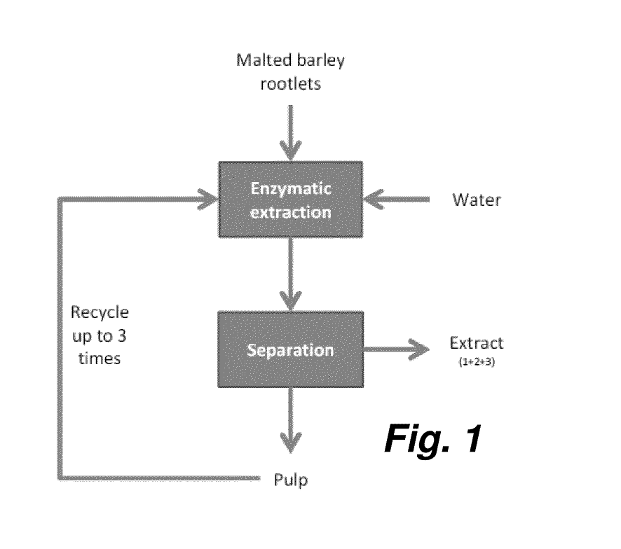

If desired, a further extractive process may be conducted. In this way, the concentration of extracted biologically active compounds may be increased by recycling the product of the enzymatic extraction into a subsequent extraction process, as shown for example in FIG. 1.

Enzymes which are indigenous to the barley malt rootlets act to produce high yields of nucleotides. Indigenous enzymes include 5′-phospho-diesterase for the production of 5′ nucleotide bases, and proteolytic enzymes for release of kinetins from their complexes with proteins (for example CK-binding proteins).

### Example 4

A liquid preparation for dispersion, namely by spray administration, was produced. The composition was formulated to further improve the taste and aroma of the product, because the liquid extract as described in Example 3 was not comfortable for use, and thereby provide a substantial contribution to the acceptability of the liquid preparation. Honey was selected as an additive for taste and the aroma was corrected by flavouring aids, although different sweeteners and/or aroma modifying agents may be used.

Composition:
Autolysate 500 ml
Honey 250 g
K-sorbate 1 g
Fruit aromaby smell

The formulation of the components was performed at room temperature. The ready-made product was packaged into spray bottles. The unit dosage in this case is approximately 12.5-13 mg/spray or 125 to 130 mg/10 sprays.

### Example 5

A gelatinous preparation, which is suitable for administration suckling for dissolution/dispersion in saliva is presented.

As described in Example 4, the product contains additives for taste and flavour correction as well as a jellying agent. There are alternative jellying agents including gelatin (animal origin) and pectin (plant origin).

Composition:
Autolysate 500 ml
Honey 200 g
Pectin 2 g
Fruit powder 5 g
K-sorbate 1 g
Ascorbic acid 5 g

The product is formulated to last for days and is packed in jars or in the form of the unit doses for delivery to the mouth.

### Example 6

The effect of liquid spray preparations as described in Example 4 on the fast recovery of athletes is shown.

The study was performed with the aim to demonstrate the effect of the preparation on the general endurance, recovery of the heart frequency, dynamic concentrations of significant metabolites involved in energetic metabolism, ventilation parameters and other indices featuring functional recovery after maximal physical efforts.

The study was performed with 12 healthy young athletes for 14 days of application. The spray was applied in 3 daily doses at 8 hours intervals, after a meal. Testing was performed before (0 day) and after application (14th day). Single dosage encompassed 10 sprays into mouth cavity—sublingual (2×), supra-lingual (2×), palatal (2×) and at buccal sights (left 2×; right 2×). Total daily dosage was approx. 5 g of the preparation.

Results: Administration of the preparation induced increased time to exhaustion during incremental running test for 30 seconds. After supplementation with the preparation for 2 weeks, maximal oxygen uptake increased for 3 ml/kg/min and ultra short-term heart rate recovery after 40, 50, 60 and 180 sec was enhanced for 5 beats on average. Intake of the preparation decreased blood lactate level for 1.2 mmol/l after 2 weeks of administration. This data demonstrated that sublingual delivery of the components in the preparation improved recovery after maxilla exercise test, particularly during 30 to 60 sec post-exercise recovery, which may be relevant for different sport disciplines and activities.

### Example 7

The effects of the preparation as described in Example 4 on immunological function of athletes are shown.

The study was performed with 38 participants randomly divided in two groups:
1. Treatment group (n=19); and
2. Placebo group (n=19).

Protocol was the same as in Example 3. The parameters were determined at the start (0 day) and at the end of intervening period (14 days).

Samples of venous blood were taken at morning in the fasting state. Additionally, samples of saliva were taken after endurance running test, for comparison with the morning non-stimulated values.

Results: Salivary Immunoglobulin A values were significantly increased after application of the contemplated preparation: 19.4±3.5 vs 25.6±5.0 ml/100 ml.

Application of the preparation for 14 days significantly diminished the drop of salivary Ig A, Ig M and lactoferrin levels after endurance tests (P<0.05). Levels of serum Ig A significantly was increased after application of the contemplated preparation: 201.4±16.9 vs 246.8±22.5μ. Count of serum Natural killer cells (NKC) was significantly increased, and consequently cytotoxic activity as well: 29.3±8.7 vs 50.4±14.5 LU.

The results demonstrate a significant potential of the composition, and in particular the nucleotides in the composition, applied in the contemplated manner in regulation of immunological functions in physically active persons.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1-50. (canceled)

51. A method of improving a response to a metabolic stress in a subject, the method comprising administering to a mucosal membrane of the subject an effective amount of an autolysate from rootlets of a barley plant of the species Hordeum, and/or an extract or a purified component of the autolysate, and thereby improve the response to the metabolic stress in the subject.

52. The method according to claim 51, wherein the response to a metabolic stress comprises recovery from a metabolic stress.

53. The method according to claim 52, wherein the metabolic stress comprises exercise induced metabolic stress.

54. The method according to claim 52, wherein the metabolic stress comprises over-training induced metabolic stress.

55. The method according to claim 51, wherein the barley plant comprises Hordeum vulgare L.

56. The method according to claim 51, wherein the mucosal membrane comprises an oral mucosal membrane.

57. The method according to claim 56, wherein the autolysate and/or an extract or a purified component thereof is administered in a form comprising one or more of a solution, a spray, a gel, a jelly, a candy, an oral disintegrating tablet, and a gum.

58. The method according to claim 51, wherein the subject comprises a human.

59. The method according to claim 51, wherein the autolysate comprises an autolysate derived from rootlets produced from malting.

60. The method according to claim 51, wherein the autolysate comprises an autolysate from sprouts and/or husks.

61. A method of improving one or more of physical performance, recovery from physical exercise and immune function in a subject, the method comprising administering to a mucosal membrane of the subject an effective amount of an autolysate from rootlets of a barley plant of the species Hordeum, and/or an extract or a purified component of the autolysate and thereby improve one or more of physical performance, recovery from physical exercise and immune function in the subject.

62. A composition for mucosal absorption, the composition comprising a barley rootlet autolysate of the species Hordeum and/or an extract or a purified component thereof.

63. The composition according to claim 62, wherein the mucosal absorption comprises oral mucosal absorption.

64. The composition according to claim 62, wherein the autolysate comprises an autolysate derived from rootlets produced from malting.

65. The composition according to claim 62, wherein the autolysate comprises an autolysate from sprouts and/or husks.

66. The composition according to claim 62, wherein the composition comprises one or more of a sweetener, a preservative, a stabiliser and a gelling agent.

67. The composition according to claim 62, wherein the composition is in a form comprising one or more of a solution, a spray, a gel, a jelly, a candy, an oral disintegrating tablet and a gum.

68. A method of improving a response to a metabolic stress in a subject, the method comprising administering an effective amount of a composition according to claim 62 to a mucosal membrane of the subject.

69. A method of improving recovery from physical exercise in a subject, the method comprising administering an effective amount of a composition according to claim 62 to a mucosal membrane of the subject.

70. A method of increasing immune function in a subject suffering from or susceptible to a metabolic stress, the method comprising administering an effective amount of a composition according to claim 62 to a mucosal membrane of the subject.
